(19) United States
(12) Patent Application Publication
(10) Pub. No.: US 2005/0036972 A1
(43) Pub. Date: Feb. 17, 2005

(54) System for managing pathogens and irritants and monitoring usage of anti-bacterial formulations

(75) Inventor: Frederick O. Cope, Columbus, OH (US)

Correspondence Address: STANDLEY LAW GROUP LLP, 495 METRO PLACE SOUTH, SUITE 210, DUBLIN, OH, 43017, US

(73) Assignee: Hy-Gene Biomedical Corporation, Columbus, OH (US)

(21) Appl. No.: 10/797,850

(22) Filed: Mar. 10, 2004

Related U.S. Application Data
(60) Provisional application No. 60/453,154, filed on Mar. 10, 2003.

(51) Int. Cl. A61K 7/06; A61K 7/11; A61K 7/075; A61K 7/08

(52) U.S. Cl. 424/70.13; 424/70.21; 424/70.24

(57) ABSTRACT

Disclosed are products that may not only kill harmful microorganisms, but may also protect the skin from harmful and irritating substances. The invention provides for formulations of antimicrobial hand soap; antibacterial topical skin barrier lotion for use on skin, over or under gloves, that moisturizes and seals out undesirable contaminants and irritants; antibacterial surface cleaner; and a validation and monitoring kit. Also included is a method for determining if employees are protected and following the protocol of application of the formulations. The products are used in combination as a system for managing microbial pathogens and irritants. The invention further provides for numerous other formulations, including, anti-itch lotion, sunscreen, spermicides, lip balm, anti-oxidant lotion, anti-aging/anti-wrinkling lotions and an insect repellent.

### FIELD OF THE INVENTION

The present invention relates to numerous specific formulations made in accordance with the process disclosed in U.S. Pat. Nos. 5,676,994, 5,882,713 and 6,238,677, which are hereby incorporated by reference, a system for managing pathogens and irritants in heath care or food preparation environments and a monitoring and validating kit and method.

### BACKGROUND OF THE INVENTION

Skin is the largest organ in the body. It is quite amazing and capable of shielding the body from just about everything it comes into contact with. Our skin is also the first line of defense against external contact and provides effective protection from the harsh elements of the environment we live and work in. The best way to soften and moisten skin is through the use of lotions and/or creams on a daily basis. It is equally important to protect our skin from the harmful exposures that are encountered on a regular daily basis, such as common household products, bodily fluids, soaps, detergents, acids, harsh chemicals, pollution in our water, toxins, bacteria, and disease-causing organisms.

Each year there are over 76 million cases of food poisoning and 300,000 hospitalizations in the United States alone. Food borne illness kills over 5,000 people each year. Over 70% of all outbreaks originate in food service operations. As many as 40% of the outbreaks are the result of poor hand washing and cross-contamination. Of the many pathogens and irritants that are found in healthcare and food service environments, clinically, the most important family is Staphylococcus. The Staphylococcus bacterium is classified into two major groups: aureus and non-aureus. S. aureus is the cause of soft tissue infections, as well as toxic shock syndrome (TSS). It can be distinguished from other members of Staphylococcus by a positive result in a coagulase test (all other species are negative). The pathogenic effects of Staphylococcus are mainly associated with the toxins it produces. Most of these toxins are produced in the stationary phase of the bacterial growth curve. The S. aureus toxin causes quick onset food poisoning that may lead to cramps and severe vomiting. Infection may be traced to contaminated meats that have not been fully cooked. These microbes also secrete leukocidin, a toxin that destroys white blood cells and leads to the formation of puss and acne. Particularly, S. aureus has been found to be the causative agent in such ailments a pneumonia, meningitis, boils, arthritis, and osteomyelitis (chronic bone infection). Most S. aureus are penicillin resistant, but vancomycin and nafcillin are known to be effective against most strains. Of the non-aureus species, S. epidermidis is the most clinically significant. This bacterium is a pathogen that is a normal resident of human skin. Those susceptible to infection by the bacterium are IV drug users, newborns, elderly, and those using catheters or other artificial appliances. Infection is easily treatable with vancomycin or rifampin.

The Streptococcus group consists of Gram-positive bacteria which appear as chains under microscopic observation. Members of Streptococcus can be aerobic, anaerobic, or microaerophilic. A grape-like appearance and a thick cell wall characterize the organisms in this group. Streptococcus pyogenes produces many toxins including hemolysins that break down blood cells. Infection can be acute and painful skin in the skin; infection may become systemic.

GROUP A—The first group includes Streptococcus pyogenes. This particular opportunistic pathogen is responsible for about 90% of all cases of pharyngitis. A common form of pharyngitis is “Strep throat” which is characterized by inflammation and swelling of the throat, as well as development of pus-filled regions on the tonsils. The infection could give rise to pneumonia. Some cases also develop into rheumatic fever. Other diseases linked to S. pyogenes are skin infections such as impetigo, cellulitis, and erysipelas.

GROUP B—The B class includes only one bacterium, S. agalactiae. For years this bacterium has been the causative agent in mastitis in cows. Currently, it has been found

to be a cause of sexually transmitted urogenital infections in females.

GROUP D—Type D Streptococcus is the next clinically important bacterium because of the multitude of diseases it is known to cause. Although many are harmless, the pathogenic strains cause complications of the human digestive tract. This group has recently been reclassified into two divisions: Enterococcus and non-Enterococcus. The Enterococci include E. faecalis, a cause of urinary tract infections, and E. faecium, a bacterium resistant to many common antibiotics. Diseases such as septicemia, endocarditis, and appendicitis have also been attributed to group D. Steptococcus and are part of the normal human fecal flora.

E. coli, the research tool of microbiology and genetics labs around the world, and it is the most encountered bacterium in the clinical laboratory. Besides being the number one cause of human urinary tract infections, E. coli has been linked to diseases in just about every other part of the body. Pneumonia, meningitis, and traveler's diarrhea are among the many illnesses that pathogenic strains of E. coli may cause. As part of the normal flora of the human intestinal tract, E. coli plays a crucial role in food digestion by producing vitamin K from undigested material in the large intestine. Pathogenic strains of E. coli, however, may cause severe cases of diarrhea in all age groups by producing a powerful endotoxin. Treating E. coli infections with antibiotics may actually place the patient in severe shock that could possibly lead to death. This is because more of the bacterium's toxin is released when the cell dies.

A Gram negative aerobic, motile, rod, Pseudomonas possesses endotoxin. It may be highly antibiotic resistant and may produce a blue-green pigment. This pathogen loves a moist environment with free proteins. It is highly opportunist, affects burn victims, diabetics, and most wounds and may cause severe respiratory tract and wound infections. It may also cause hot tub dermatitis.

Hepatitis viruses, A, B, and C cause both acute and chronic infections in man. An unusual feature is the prolonged viremia, lasting for up to several months in acute infections and for many years (even for life)-in chronic infections. Hepatitis is transmitted through human fluids and contact.

Among the most common causes of skin irritation, disease, and injury are over-exposure to extreme temperatures or radiation, chemical substances (such as solvents, alkalis, acids, hand detergents, etc.) biological substances (body fluids, viruses, bacteria, fungi), and plant substances (such as the oil in poison ivy, oak, and sumac). Whenever possible, it is wise to avoid contact with such irritants. If one works with or around substances known to cause skin problems, their company should help identify those hazards, advise them about safe work practices, and provide personal protective equipment to help ensure that their skin remains healthy and well protected.

Accordingly, there is a need for a product and system for managing pathogens and irritants in health care or food preparation environments.

### SUMMARY OF THE INVENTION

Accordingly, the present invention is to novel products that may not only kill harmful microorganisms, but may also protect the skin from harmful and irritating substances.

The invention provides for formulations of an antimicrobial hand soap, an antibacterial topical skin barrier lotion for use on skin, over or under gloves, that moisturizes and seals out undesirable contaminants and irritants, antibacterial surface cleaner and a validation and monitoring kit and method for determining if employees are protected and following the protocol of application. The products are used in combination as a system for managing microbial pathogens and irritants.

The formulations of the present invention fight microbial pathogens both instantly and for 4-6 hours after application. The formulations of the present invention further provide protection even when washed 2-3 times with mild soap and/or alcohol-based solvents.

The system of the present invention is formulated with the soap, lotion and surface cleaner of the present invention containing a “cocktail” of antimicrobials that attack and kill pathogens in different ways, making it unlikely that pathogens will develop resistance to these products.

The invention further provides for numerous other formulations, including, anti-itch lotion, sunscreen, spermicides, lip balm, anti-oxidant lotion, anti-aging/anti-wrinkling lotions and an insect repellent.

### DESCRIPTION OF THE DRAWINGS

FIG. 1 is a schematic of a method of making the formulations of the present invention in accordance with the method disclosed in U.S. Pat. Nos. 5,676,994, 5,882,713 and 6,238,677;

FIG. 2 is a scanning electron micrograph showing generally a product of the present invention made in accordance with the method disclosed in U.S. Pat. Nos. 5,676,994, 5,882,713 and 6,238,677;

FIG. 3 is a chart showing the different processes that result in different formulations of products of the present invention;

FIG. 4 is a bar graph that discloses respondents rating of greasiness of a product of the present invention;

FIG. 5 is a bar graph that discloses respondents rating of texture of a product of the present invention;

FIG. 6 is a bar graph that discloses respondents rating of stickiness of a product of the present invention;

FIG. 7 is a chart that rates % hydration versus a sebum reading for a product of the present invention versus other commercial lotions;

FIG. 8 is a line graph showing the durability rating of a product of the present invention;

FIG. 9 is a bar graph that shows when crystal violet is applied to bare skin (the control) there is 100% staining but when applied to skin coated with one of several commercially available products the amount of staining is reduced to between about 40% to about 50%.

FIG. 10 is a bar graph that shows when crystal violet is applied to bare skin (the control) there is 100% staining but when applied to skin coated with a product of the present invention there is only about 20% staining that occurs.

FIG. 11 is a bar graph that exemplifies the resistance of a product of the present invention to water washings.

FIG. 12 is a bar graph that shows the resistance of a product of the present invention to washing with alcohol.

FIG. 13 is a bar graph that exemplifies the staying power and thus retention of killing capacity of a product of the present invention even after washing with soap and water.

FIG. 14 is a bar graph that contrasts the removal of a commercially available product with soap and water.

FIG. 15 is a line graph that rates skin moisture after an application of a product of the present invention;

FIG. 16 is a bar graph showing hydrocortisone penetration levels on cadaver skin after 24 hours of two products of the present invention compared to a product in the prior art;

FIG. 17 is a bar graph showing percent kill rates as a function of time of a product of the present invention against Staphylococcus Aureus;

FIG. 18 is a bar graph showing percent kill rates as a function of time of a product of the present invention against Methicillin resistant Staphylococcus Aureus;

FIG. 19 is a bar graph showing percent kill rates as a function of time of a product of the present invention against Pseudomonas Aeruginosa;

FIG. 20 is a bar graph showing percent kill rates as a function of time of a product of the present invention against E. Coli;

FIG. 21 is a bar graph showing percent kill rates as a function of time of a product of the present invention against Streptococcus Pyrogenes;

FIG. 22 is a bar graph showing percent kill rates as a function of time of a product of the present invention against HIV;

FIG. 23 is a bar graph showing percent kill rates as a function of time of a product of the present invention against Hepatitis B & C; and

FIG. 24 is a bar graph showing percent kill rates, as a function of time, of a product of the present invention against other pathogens.

FIG. 25 is a plot of clinical irritation scores of skin sites of 4 human volunteers subjected to 24 hours occluded Finn chambers containing sterile saline-soaked wafers (vehicle), or 1%-SLS, 2%-SLS and 4%-SLS.

FIG. 26 is a plot showing the prevention of ICD due to 24-hour occlusion of human skin with SLS by a 15 minute pre-treatment with two different starch-oil composite formulations, DC-64A and DC-64E. The results are plotted as frequency (n/T) of skin sites with a given clinical score versus clinical irritation score.

FIG. 27 is a bar graph showing the prevention of irritation-induced skin hydration by pre-treatment of skin sites occluded for 24 hours with 1% SLS or 2% SLS versus water, 1% SLS or 2% SLS alone. The abscissa plots the % Hydration normalized as the ratio of the treatment response to water treatment response.

FIG. 28 is a bar graph showing bacterial growth counts on instruments in a general hospital setting when the instruments have not been treated, have been treated with alcohol and have been treated with a spray surface cleaner formulation of the present invention.

FIG. 29 is a bar graph showing corrected bacterial growth counts on personnel hands in a hospital setting when hands have not been treated, have been treated with alcohol and have been treated with a spray surface cleaner formulation of the present invention.

FIG. 30 is a bar graph showing uncorrected bacterial growth counts on personnel hands in a hospital setting when hands have not been treated, have been treated with alcohol and have been treated with a spray surface cleaner formulation of the present invention.

FIG. 31 is a bar graph showing bacterial growth counts on surfaces in a surgical setting when the surfaces have not been treated, have been treated with alcohol and have been treated with a spray surface cleaner formulation of the present invention.

FIG. 32 is a bar graph showing corrected bacterial growth counts on floor surfaces in a general hospital setting when the floors have not been treated, have been treated with alcohol and have been treated with a spray surface cleaner formulation of the present invention.

FIG. 33 is a bar graph showing corrected bacterial growth counts on floor surfaces in a surgical setting when the floors have not been treated, have been treated with alcohol and have been treated with a spray surface cleaner formulation of the present invention.

FIG. 34 is a bar graph showing uncorrected bacterial growth counts on floor surfaces in a general hospital setting when the floors have not been treated, have been treated with alcohol and have been treated with a spray surface cleaner formulation of the present invention.

FIG. 35 is a bar graph showing uncorrected bacterial growth counts on floor surfaces in a surgical setting when the floors have not been treated, have been treated with alcohol and have been treated with a spray surface cleaner formulation of the present invention.

### DESCRIPTION OF EXAMPLE EMBODIMENTS

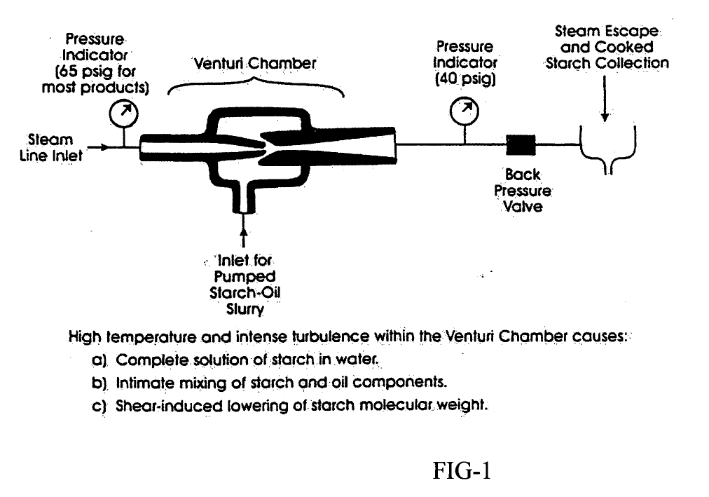

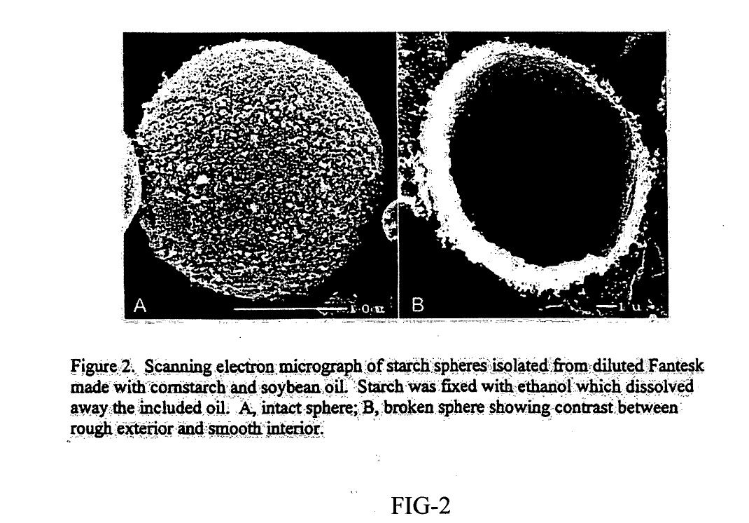

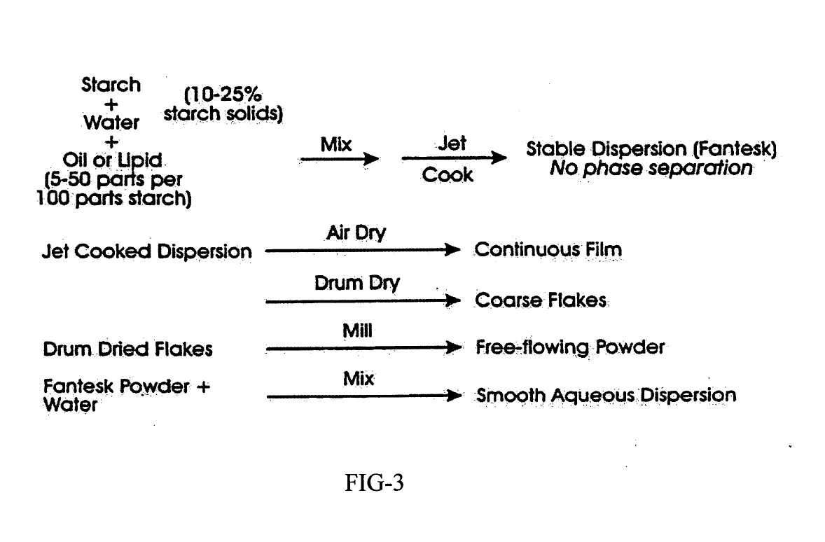

Two different topical application formulations of the present invention were prepared and were designated as formulations DC-64A and DC-64E. The former is a lotion prepared by jet-cooking a mixture containing 100 parts of waxy cornstarch to 10 parts of petrolatum jelly. The later lotion was prepared by jet-cooking a mixture containing 100 parts of waxy cornstarch to 50 parts of petrolatum jelly. The jet-cooked emulsions so formed were drum-dried, milled to a fine powder (<0.5 mm) and stored under low (<5%) humidity. The powders were hydrated to give a final of 20% solids (w/v) with sterile de-ionized water. The resulting pourable lotions were used directly. A close-up of the product of the present invention is shown in FIG. 2. All other chemicals used in this study were purchased from Sigma Chemical Co., (St. Louis, Mo.). FIG. 1 shows the process utilized to make the products of the present invention and FIG. 3 shows the production cycle.

Following is a portion of the Material Safety Data Sheet (MSDS) for the antibacterial lotion of the present invention showing the ingredients with preferred weight percent and in parenthesis the range of weight percent that is possible to use:
Purified water—87.75% (80.0-99.5%)
Waxy Starch OR other Polysaccharide (natural or synthetic)—7% (0.5-15.0%)
Petrolatum OR other mineral oil—3% (0.125-12.0%)
Other Plant/vegetable oils or extracts—0.25% (0.01-8.0%)
Pectin OR other viscosity stabilizer—0.5% (0.01-3.0%)
Xanthan OR other viscosity enhancer—0.5% (0.01-3.0%)
d-α-tocopherol or other antioxidant or stabilizer—0.15% (0.01-5.0%)
Benzoic Acid—0.1%—OPTIONAL (0.01-1.0%)
Boric Acid—0.1%—OPTIONAL (0.01-1.0%)
MgCl2-0.05% (0.01-1.0%)
Methyl Paraben—0.1% (0.01-1.0%)
Propyl Paraben—0.1% (0.01-1.0%)
Citricidal—0.1% (0.01-3.0%)
Benzalkonium Cl OR other quaternary ammonium compound(s) in combination of the indicated concentration—0.35% (0.01-2.0%)
Tri-N-Butyl PO₄-0.125% (0.01-1.0%)
May contain—benzoyl peroxide—1.0% @q.s. (0.05-3.0%)

There were a total of 26 healthy volunteers of both genders: four subjects in Study A (dose ranging pilot evaluation), and total of 22 subjects in Study B (main evaluation. Inclusion criteria: Subjects completed a consent form and had a minimum age of 18. Both men and women were recruited. Subjects could have medical conditions controlled by medication, provided they did not receive any of the medications listed under exclusions. Exclusion criteria: Women must not be pregnant, and must be practicing contraception if capable of becoming pregnant. Subjects with any active inflammatory acneiform or infectious disease process which is not controlled by medication—subjects with other precluding active skin conditions on their back-subjects with a recent history of eczema, or psoriasis, to prevent reactivation of skin diseases are excluded. History of allergy to tape, detergents, immunosuppressive conditions including cancer chemotherapy within the last 6 months, HIV, hepatitis and other precluding conditions as the physician may deem appropriate may be excluding factors. In addition, medications that are cause for exclusion include prednisone, diuretics, topical steroid application to the back within the last month, or, in the opinion of the physician, other precluding factors. Lastly, persons with pulmonary sensitivity or hypersensitivity to detergents were excluded from the study.

Patch Tests with Sodium Lauryl Sulfate (SLS)

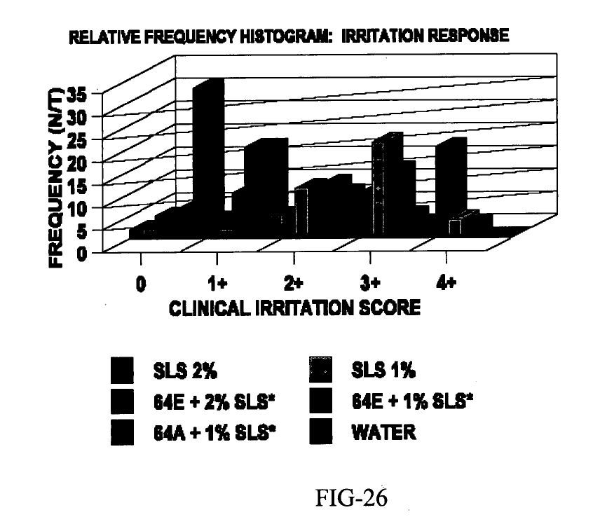

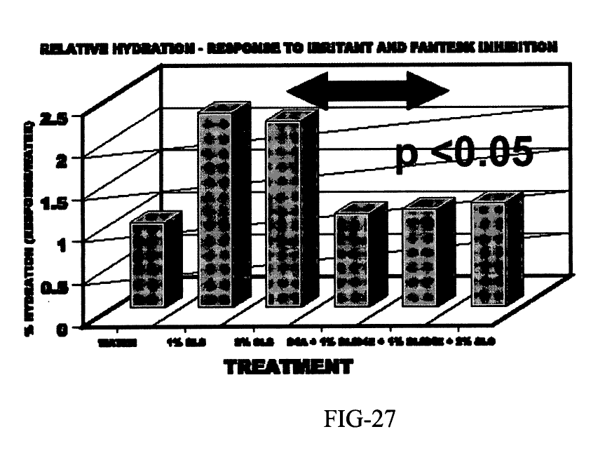

FIGS. 26 and 27 document some of the results of this testing. Irritant patch tests with SLS, sodium lauryl sulfate (a known irritant) were performed with and without prior treatment with lotion of the present invention. Irritation was scored visually and by use of skin capacitance Corneometer, Model CM825, Courage and Kahazaka, Cologne, Germany). The variables were concentration of SLS, and absence or presence of pre-treatment with lotion. The standard model of ICD (irritant contact dermatitis) used in these studies is the occluded irritant patch test to the detergent sodium lauryl sulfate (SLS). Occluded irritant patch testing of humans with SLS is a well-documented procedure. SLS irritant patch tests have been used to evaluate the efficacy of protective barrier creams SLS was applied as a (1,2, & 4%) aqueous solution for 24 hr in an “extra large” Finn chamber, with a capacity of 0.3 ml and a diameter of 18 mm. High purity >99% SLS was used dissolved in USP grade water. SLS was loaded onto an appropriate size filter disc, and then loaded into the Finn Chamber (Epitest, Hermal Pharmaceutical Laboratories, Oak Hill, N.Y.). The Finn Chambers were then attached to the skin of the back with tape (Epitest). Subjects were not able to get the test site wet for the 24 hrs the patch was applied. Test chambers were removed 24 hrs later and irritation was graded visually. Lotion was applied to skin of the back 15 minutes prior to the SLS Finn chamber. The target area of the back was designated with a circular template 2.0 cm in diameter. The lotion was measured out in a 1.0 ml pipette. A total of 0.3 ml of lotion was measured on to this target area and applied over the 2.0 cm circle with a gloved finger.

Visual Grading Scale for Irritation

Erythema, edema, and epidermal changes were graded on a scale from 0, none, to 4+erythema with edema, and epidermal erosions. Visual scales have been shown to result in statistically significant response differences in SLS irritant patch test studies of barrier cream protection.

Skin Moisture Evaluation of Patch Test Sites

The Corneometer (Model CM825, Courgage+Khazaka, Cologne Germany) has been shown to detect changes in skin capacitance following occlusive patch testing of human skin with 2% SLS (10), and relate to the hydration status of the epidermis. Within 5 minutes of removing the occlusive patch each subject had all 12 of their treated skin sites measured with the Corneometer instrument, The probe was applied repetitively a total of 10 times to each skin site and the mean ±1×S.E. calculated by the vendor supplied software program. Skin hydration values are the instrument calibrated values with a background reading (dry paper towel) equal to 5, and water-soaked paper towel equal to 110. Extensive validation studies were conducted to verify the proper environmental conditions for subject testing that produced statistically reliable values. These included a 30 minute pre-adaptation of the subject to controlled temperature (24-25° C. and controlled relative humidity (50-60%) environment Under these conditions subjects gave repeatable yet idiosyncratic baseline readings.

The study was divided into a preliminary phase, part (A), and a final phase, part (B). Part (A) was a pilot study for determining the optimal range of SLS for irritant dermatitis studies. Part (B), main evaluation, tested the ability of the lotion to protect against SLS irritation. The solutions were coded and the technician was blinded to the concentration of SLS. But it was not be possible to blind the technician to the skin sites receiving the lotion. For this reason, the person evaluating the patch tests was blinded to the treatments, and did not have knowledge of the test solution application patterns. Three persons read the results and estimations of response were compared.

Part A: Subjects received the below treatments to normal skin on their back. The lotion (0.3 ml) was applied to the skin first to an area of 2 cm in diameter. Fifteen minutes latter, 0.3 ml of SLS was applied under a 18 mm Finn chamber. The following treatment groups were randomized according to a prospectively randomized matrix of assignments: 1&2) SLS-1.0%, 3 &4) SLS-2.0%, 5 & 6) SLS-4%, and 7 & 8) SLS-Vehicle (water). The subjects did not shower or get the test site wet for 24 hours. After 24 hours the Finn chambers were removed and irritation was graded by visual scale (see Table 1 below). All test sites were documented with photography. The above evaluation was used to determine a concentration of SLS that provided an irritation response of 2 or 3 without lotion pretreatment.

TABLE 1
VISUAL SCALE FOR GRADING IRRITATION RESPONSES

0 = No alteration, no erythema
1+ = Slight erythema distributed over most of treatment site
2+ = Distinct erythema with slight elevation (edema) over entire site of
chamber
3+ = Erythema associated with elevation (edema), as well as vesiculation
of epidermis
4+ = Erythema with epidermal erosions

Part B: Part B utilized two concentrations of SLS as determined above. There were 22 subjects enrolled. To control for intra-subject variation, each subject was tested with replicates for a total of 12 Finn chambers per subject. All Finn chambers were applied to skin on the main trunk of the back between the waist and the clavicle. The following treatment groups were randomized according to a prospectively randomized matrix of assignments: 1)SLS-1%, 2) pre-treat with DC-64E followed by SLS-1%, 3) SLS-2%, 4) pre-treat with DC-64E followed by SLS-2%, 5) pre-treat with DC-64A followed by SLS-1%, 6) pre-treat with DC-64A followed by SLS Vehicle. The lotion (0.3 ml) was applied to the skin first (with a swab) to a circular area of 2 cm in diameter. Fifteen minutes subsequent, 0.3 ml of SLS was applied under an 18 mm Finn chamber as described below. A matrix supplied to the technician applying the Finn Chambers randomized all treatment groups. The subjects were not allowed to shower or get the test site wet for 24 hours. After 24 hours the Finn chambers were removed and irritation was graded by visual scale as well as skin capacitance (Corneometer). The investigator(s) evaluating the test sites with the visual scale and the Corneometer were blinded to the treatment sites. All test sites were documented with photography.

Statistical Analysis

Both the visual scoring system and the Corneometer measurements have been shown to provide statistically significant sensitivity in the irritant patch testing using SLS. A previous study of protective barrier creams and SLS patch testing was able to show statistically significant results using the visual scoring system with 10 subjects. Study B used 22 subjects and as such, provides discrimination to the study (based on a 20% difference of irritation) of P<0.05 and an a of >80%. Visual scores are non-parametric data were analyzed by Chi-square in a paired comparison between treated and non-treated sites. The Corneometer data were analyzed as ANOVA, pair-wise T-test comparison between treated and non-treated sites.

Adverse Events

Throughout the duration of the study, the Principal Investigator monitored each subject for evidence of procedural intolerance and for the development of clinical evidence of undue adverse events. It should be stated that adverse event were expected in this study due to reactions to the SLS. All adverse events that occurred during the course of the study were recorded on the appropriate case report form, photographed, and followed to satisfactory resolution. If an unexpected reaction occurred or, if in the opinion of the investigator, any untoward response to the procedure occurred, regardless of treatment assignment, the investigator provided appropriate medical action. In three cases there were untoward reactions to the application of the materials, and these subjects were dropped by the recommendation or by subject choice. Any test or evaluation performed at any time during the study demonstrating significant changes from the corresponding pre-study test or evaluation was followed at medically appropriate intervals until resolution of the abnormality. There were no serious or unexpected adverse events.

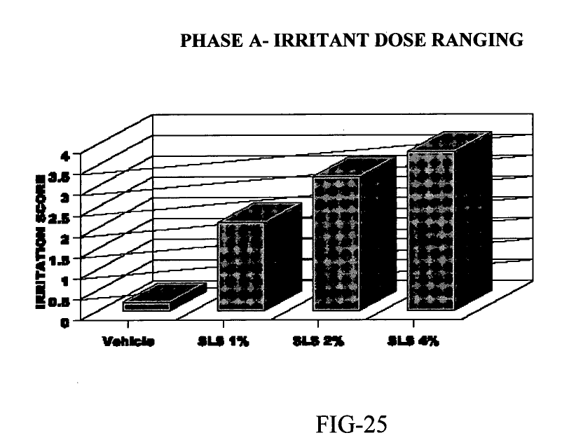

The SLS dose-ranging study (Part A) determined that the concentrations of SLS of 1% and 2% were sufficient to elicit an erythema and erosion in the midrange of the clinical evaluation scale, while a concentration of 4% was too severe to be employed for further studies (FIG. 25). Two concentrations of SLS which bracket the irritation response of 2-3 were chosen for the main evaluation in Part (B). As expected, there was an linearly increasing severity of skin response with increasing dose of SLS (correlation coefficient r=0.93, p<0.05).

A summary of results for all subjects of the main evaluation of clinical irritation assessments portion of the study (Part B) are presented in Table 2 shown below. Of the 22 original subjects, only 20 completed the protocol. Columns 1 and 2 represent the two independent skin sites (left and right sides of back) that were treated with 1% SLS. The mean clinical irritation score was 2.6±0.8 and 2.5±1.1. This compares with 0.3±0.6 and 0.2±0.4 for the two independent water-treated skin sites (columns 5 &6). Similarly for the skin sites treated with 2% SLS, the clinical irritation scores were 3.2±1.0 and 2.9±1.4(columns 3 &4). These data confirm the increased severity of the skin response for skin sites treated with 2% SLS relative to 1% SLS and water. Table 2 also presents tabular data for the effect of a 15-min pre-treatment of skin sites with lotion (DC-64A) prior to 24 hour occlusion under with either 1% SLS (columns 7 & 8) versus 2% SLS (columns 9 & 10), respectively. The mean clinical score for DC-64A-1% SLS treatment group was 1.3±01.0 and 1.3±0.8. These results demonstrate the skin barrier protective effect of lotion DC-64A against insult by 1% SLS. Likewise pre-treatment of skin sites with a different lotion DC-64E prior to 1% SLS again produced a highly significant protective effect (mean clinical scores of 1.5±0.9 and 1.5±0.9). The protective effect of lotion DC-64E was also observed against 2% SLS (mean clinical score 2.0±1.3 and 1.8±1.3). The last row of Table 2 summarizes the mean clinical scores for each treatment group rounded to nearest whole integer. The mean score now reflects the elimination of one outlier value, (subject 12).

TABLE 2
Prevention of Irritation Due to Skin Occlusion with a Surfactant by DC-64A and
DC-64E Lotions (Clinical Irritation Score)

| | SLS- | SLS- | SLS- | SLS- | | | 64A+1% | 64A+1% | 64E+1% | 64E+1% | 64E+2% | 64E+2% |
|---|---|---|---|---|---|---|---|---|---|---|---|---|
| M/I | 1% | 1% | 2% | 2% | Water | Water | sls | sls | sls | sls | sls | sls |
| 1/ | 2 | 3 | 4 | 4 | 0 | 0 | 2 | 3 | 0 | 3 | 3 | 2 |
| afh | | | | | | | | | | | | |
| 2/ | 3 | 3 | 4 | 4 | 0 | 0 | 0 | 0 | 1 | 1 | 1 | 3 |
| hjh | | | | | | | | | | | | |
| 3/ | 2 | 1 | 4 | 4 | 1 | 0 | 2 | 1 | 1 | 1 | 1 | 2 |
| sds | | | | | | | | | | | | |
| 4/trf | 3 | 4 | 4 | 4 | 0 | 0 | 2 | 1 | 2 | 2 | 3 | 0 |
| 6/aam | 2 | 2 | 0 | 0 | 2 | 1 | 0 | 1 | 3 | 1 | 3 | 3 |
| 7/n-r | 3 | 3 | 3 | 1 | 0 | 0 | 1 | 2 | 2 | 1 | 2 | 2 |
| 8/ | 3 | 3 | 3 | 1 | 0 | 1 | 0 | 1 | 1 | 0 | 1 | 0 |
| paw | | | | | | | | | | | | |
| 9/jes | 2 | 3 | 3 | 4 | 0 | 0 | 1 | 0 | 1 | 1 | 4 | 3 |
| 11/ | 3 | 3 | 3 | 3 | 0 | 0 | 3 | 2 | 2 | 2 | 3 | 3 |
| dal | | | | | | | | | | | | |
| 12/ktp | 0 | 0 | 2 | 1 | 0 | 0 | 2 | 1 | 1 | 1 | 1 | 1 |
| 13/sms | 2 | 3 | 4 | 4 | 0 | 0 | 1 | 1 | 2 | 2 | 3 | 4 |
| 14/ | 2 | 2 | 2 | 3 | 0 | 1 | 0 | 1 | 0 | 1 | 1 | 0 |
| jah | | | | | | | | | | | | |
| 15/ | 3 | 4 | 2 | 3 | 0 | 0 | 1 | 1 | 1 | 1 | 3 | 0 |
| dkl | | | | | | | | | | | | |
| 16l/sg | 2 | 1 | 4 | 1 | 2 | 1 | 1 | 1 | 3 | 3 | 1 | 2 |
| 17/ | 3 | 3 | 3 | 2 | 0 | 0 | 3 | 2 | 1 | 3 | 1 | 2 |
| ljl | | | | | | | | | | | | |
| 18/ | 3 | 2 | 4 | 4 | 0 | 0 | 3 | 2 | 3 | 2 | 3 | 3 |
| d-s | | | | | | | | | | | | |
| 19/kkg | 4 | 2 | 4 | 4 | 0 | 0 | 1 | 2 | 1 | 1 | 3 | 0 |
| 20/ttm | 3 | 1 | 3 | 3 | 0 | 0 | 1 | 2 | 1 | 0 | 0 | 2 |
| 21/ | 3 | 3 | 4 | 4 | 0 | 0 | 1 | 0 | 2 | 2 | 1 | 1 |
| lea | | | | | | | | | | | | |
| 22/ | 3 | 4 | 4 | 4 | 0 | 0 | 1 | 2 | 2 | 2 | 4 | 3 |
| tal | | | | | | | | | | | | |
| X ± sd | 2.4 ± | 2.5 ± 1.1 | 3.2 ± 1.0 | 2.9 ± 1.4 | 0.3 ± 0.6 | 0.2 ± 0.4 | 1.3 ± 1.0 | 1.3 ± 0.8 | 1.5 ± 0.9 | 1.5 ± 0.9 | 2.0 ± 1.3 | 1.8 ± 1.3 |
| N = 20 | 1.0 | | | | | | | | | | | |
| Score | 2 | 3 | 3 | 3 | 0 | 0 | 1 | 1 | 2 | 2 | 2 | 2 |

M, Subject matrix code;
I, initials;
64A+1% sls, 64E−1% sls, 64E−2% sls = Derm-Care ™ lotions applied prior to SLS

Increased Skin Hydration Induced by SLS Treatment

Table 3 shown below presents a summary of the skin capacitance readings for all 20 subjects. Based on previous studies and our own internal controls, we interpret elevated skin capacitance reading as evidence of increased skin hydration. Columns 1 and 2 list the subject code numbers and initials, respectively. Columns B1 and B2 represent the baseline skin capacitance values for each subject taken along the left and the right sides of the sacral midline 24 hours prior to the application of occlusive patches. Although, it is clear that there is inter-subject variation with values ranging between 46 and 76, the intra-subject variation is less than 5% of the mean, and there is remarkable congruity between adjacent left and right side duplicates (mean left=58±8; mean right=58±8). For the water-treated skin sites (columns 5&6), the mean values were 47±10 and 46±9). Note that two outliers were dropped (subjects 6 & 13). The mean water-treated values are less than baseline but are not statistically different (P>0.06) for 1% SLS-treated skin sites, the mean values were 104±14 and 98±19. Five subjects were dropped as outliers due to anomalously low values. In each case registration of these anomalous reading correlated with the appearance of dead epidermal tissue covering an otherwise eroded skin site. For 2% SLS-treated skin sites the mean values were 100±20 and 94±29. Again, five subjects were eliminated because of anomalous reading similar in nature to the 1% SLS group. In summary, 1% and 2% SLS treated skin sites displayed significantly higher skin hydration levels.

TABLE 3
Prevention of Skin Hydration Induced by SLS by DC-64A and DC-64E Lotions
(Corneomter readings)

| | | | | SL— | SL— | SL— | SL— | |
|---|---|---|---|---|---|---|---|---|
| | | B | B | 1% | 1% | 2% | 2% | W |
| M | I | L | R | 1 | 2 | 3 | 4 | 5 |
| 1 | afh | 54 ± 2 | 67 ± 3 | 114 ± 8 | 116 ± 11 | 121 ± 10 | 122 ± 10 | 62 ± 6 |
| 2 | hjh | 49 ± 3 | 58 ± 2 | 55 ± 3 | 55 ± 6 | 101 ± 3 | 108 ± 15 | 47 ± 3 |
| 3 | sds | 61 ± 4 | 53 ± 3 | 25 ± 3 | 38 ± 5 | 114 ± 7 | 96 ± 16 | 59 ± 5 |
| 4 | trf | 55 ± 2 | 59 ± 1 | 90 ± 14 | 109 ± 9 | 115 ± 6 | 121 ± 0 | 49 ± 3 |
| 6 | aam | 76 ± 4 | 74 ± 2 | 81 ± 4 | 63 ± 5 | 76 ± 5 | 59 ± 4 | 98 ± 6 |
| 7 | n-r | 53 ± 2 | 61 ± 3 | 82 ± 11 | 99 ± 11 | 74 ± 10 | 45 ± 15 | 25 ± 1 |
| 8 | paw | 55 ± 2 | 52 ± 1 | 95 ± 10 | 104 ± 8 | 52 ± 4 | 33 ± 4 | 46 ± 4 |
| 9 | jes | 58 ± 2 | 52 ± 2 | 109 ± 12 | 117 ± 8 | 87 ± 8 | 66 ± 6 | 45 ± 6 |
| 11 | dal | 58 ± 2 | 59 ± 4 | 120 ± 1 | 121 ± 2 | 84 ± 6 | 93 ± 8 | 42 ± 4 |
| 13 | sms | 59 ± 3 | 64 ± 2 | 118 ± 3 | 120 ± 1 | 121 ± 1 | 121 ± 1 | 113 ± 5 |
| 14 | jah | 50 ± 1 | 47 ± 3 | 117 ± 3 | 100 ± 8 | 44 ± 4 | 101 ± 6 | 47 ± 2 |
| 15 | dkl | 76 ± 1 | 70 ± 2 | 104 ± 9 | 102 ± 6 | 67 ± 4 | 92 ± 11 | 52 ± 3 |
| 16 | lsg | 62 ± 2 | 58 ± 2 | 33 ± 6 | 41 ± 7 | 121 ± 2 | 30 ± 2 | 64 ± 6 |
| 17 | ljl | 66 ± 10 | 67 ± 4 | 109 ± 5 | 75 ± 5 | 47 ± 5 | 87 ± 6 | 36 ± 2 |
| 18 | d-s | 56 ± 2 | 56 ± 1 | 116 ± 10 | 106 ± 7 | 113 ± 7 | 113 ± 8 | 45 ± 2 |
| 19 | kkg | 46 ± 3 | 43 ± 2 | 81 ± 5 | 70 ± 10 | 47 ± 8 | 98 ± 12 | 39 ± 6 |
| 20 | ttm | 55 ± 2 | 54 ± 2 | 116 ± 3 | 43 ± 9 | 112 ± 1 | 102 ± 6 | 34 ± 3 |
| 21 | lea | 58 ± 4 | 55 ± 1 | 38 ± 9 | 81 ± 12 | 68 ± 8 | 33 ± 6 | 46 ± 5 |
| 22 | tal | 60 ± 1 | 63 ± 3 | 67 ± 7 | 73 ± 6 | 109 ± 15 | 119 ± 0 | 54 ± 2 |
| 23 | ktp | 53 ± 3 | 56 ± 2 | 104 ± 15 | 82 ± 8 | 100 ± 4 | 76 ± 13 | 55 ± 6 |

| | W | 64A−1% | 64A−1% | 64E−1% | 64E−1% | 64E−2% | 64E−2% |
|---|---|---|---|---|---|---|---|
| M | 6 | 7 | 8 | 9 | 10 | 11 | 12 |
| 1 | 55 ± 6 | 52 ± 5 | 76 ± 5 | 50 ± 3 | 88 ± 4 | 67 ± 5 | 84 ± 10 |
| 2 | 57 ± 2 | 58 ± 3 | 53 ± 1 | 45 ± 3 | 43 ± 4 | 72 ± 5 | 53 ± 8 |
| 3 | 46 ± 4 | 57 ± 5 | 38 ± 8 | 47 ± 3 | 24 ± 2 | 36 ± 6 | 41 ± 7 |
| 4 | 47 ± 5 | 54 ± 4 | 59 ± 3 | 55 ± 6 | 57 ± 6 | 48 ± 8 | 41 ± 4 |
| 6 | 65 ± 4 | 67 ± 7 | 72 ± 4 | 78 ± 6 | 97 ± 11 | 104 ± 11 | 83 ± 5 |
| 7 | 33 ± 2 | 32 ± 3 | 38 ± 6 | 25 ± 1 | 30 ± 3 | 52 ± 7 | 59 ± 5 |
| 8 | 42 ± 5 | 45 ± 3 | 30 ± 4 | 39 ± 9 | 55 ± 6 | 49 ± 7 | 45 ± 5 |
| 9 | 46 ± 4 | 57 ± 10 | 30 ± 5 | 46 ± 4 | 49 ± 4 | 58 ± 6 | 76 ± 13 |
| 11 | 61 ± 5 | 90 ± 6 | 96 ± 9 | 103 ± 12 | 75 ± 11 | 79 ± 7 | 65 ± 4 |
| 13 | 75 ± 5 | 118 ± 5 | 121 ± 6 | 112 ± 6 | 113 ± 11 | 90 ± 7 | 110 ± 10 |
| 14 | 38 ± 13 | 25 ± 1 | 28 ± 2 | 36 ± 3 | 30 ± 2 | 29 ± 4 | 34 ± 11 |
| 15 | 43 ± 5 | 30 ± 8 | 29 ± 3 | 42 ± 5 | 65 ± 24 | 107 ± 18 | 66 ± 5 |
| 16 | 50 ± 3 | 25 ± 1 | 30 ± 5 | 27 ± 2 | 52 ± 5 | 33 ± 4 | 38 ± 9 |
| 17 | 50 ± 6 | 71 ± 5 | 30 ± 2 | 39 ± 2 | 71 ± 7 | 32 ± 2 | 63 ± 8 |
| 18 | 43 ± 3 | 79 ± 5 | 79 ± 8 | 75 ± 9 | 81 ± 5 | 99 ± 14 | 119 ± 4 |
| 19 | 28 ± 1 | 30 ± 3 | 25 ± 3 | 39 ± 7 | 37 ± 6 | 40 ± 4 | 36 ± 2 |
| 20 | 34 ± 3 | 29 ± 4 | 46 ± 1 | 45 ± 4 | 63 ± 2 | 30 ± 5 | 59 ± 4 |
| 21 | 41 ± 1 | 47 ± 3 | 29 ± 5 | 39 ± 6 | 61 ± 4 | 30 ± 4 | 41 ± 6 |
| 22 | 55 ± 1 | 33 ± 3 | 64 ± 7 | 46 ± 11 | 73 ± 4 | 74 ± 12 | 73 ± 4 |
| 23 | 52 ± 4 | 77 ± 5 | 47 ± 7 | 32 ± 2 | 71 ± 19 | 45 ± 8 | 35 ± 6 |

M = matrix number;
I = initials;
B = baseline;
SL = SDS;
W = water;
64A/64E lotions

Prior treatment of skin sites with lotion DC-64A significantly reduced the mean skin hydration levels in both duplicate skin sites (48±18 and 47±21). Two subjects (11 and 13) were eliminated as outliers from these mean hydration values. Pre-treatment with formulation DC-64E also produced a significant reduction in skin hydration at duplicate skin sites caused by 1% SLS (45±14 and 45±14). Again the same two outliers were dropped (subjects 11 and 13). Finally, pre-treatment with formulation DC-64E also significantly reduced the elevated skin hydration at duplicate treated sites caused by 2% SLS (54±22 and 55±17).

In summary, the results show that application of starch-oil composite lotion of the present invention has excellent skin barrier properties. The unique oil-in-water dispersion process forms a protective polymer film on intact human skin. It is assumed that this protective film prevents water-soluble irritants such as SLS from penetrating into the outer layers of the skin even under conditions of occlusion. The protective role of the incorporated and dispersed petrolatum droplets is based on its demonstrated ability to keep skin moisturized in areas that are in direct skin contact. The role of the polysaccharide matrix is to form a protective hydrating film that absorbs moisture and shields the skin contact area from penetration by water-soluble irritants. Without the skin barrier skin sites that were treated with SLS had significantly elevated clinical irritation scores and elevated skin hydration. Together, the clinical response results and the skin hydration results provide strong evidence for a protective role for starch-oil composites as skin barrier lotions. Based on the above results we also suggest that starch-oil composites such as lotions of the present invention have the potential to prevent adverse skin reactions now encountered with many current skin care and wound care products.

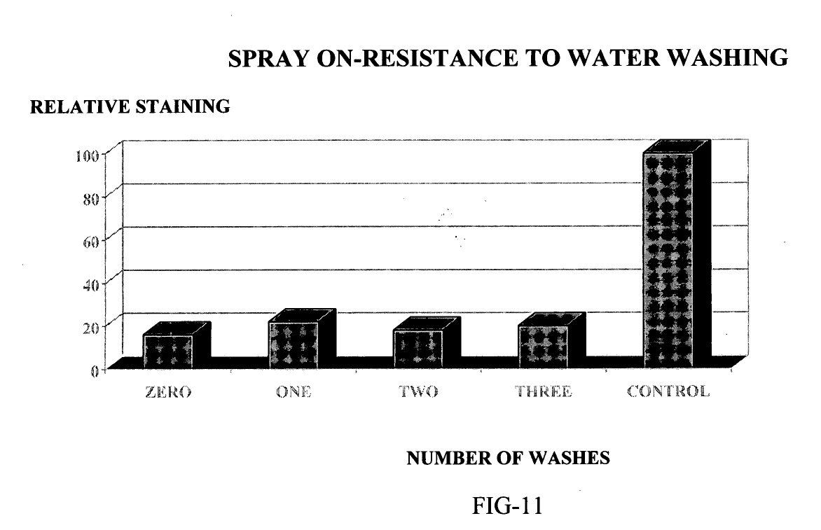

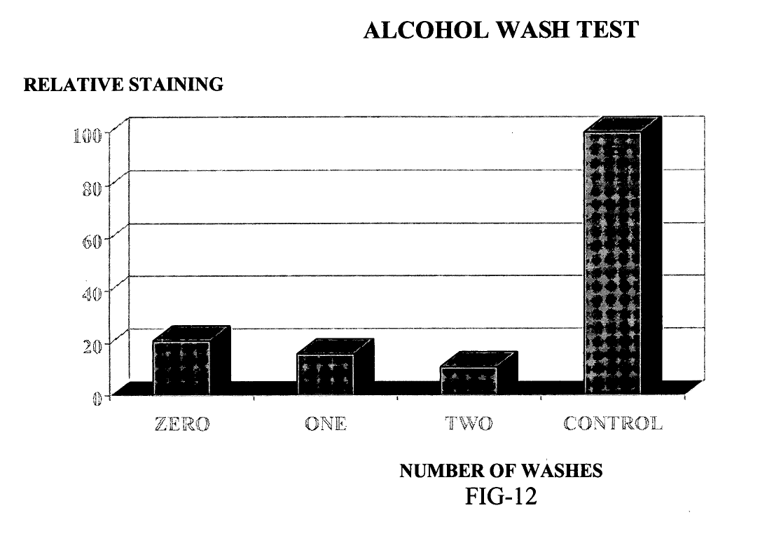

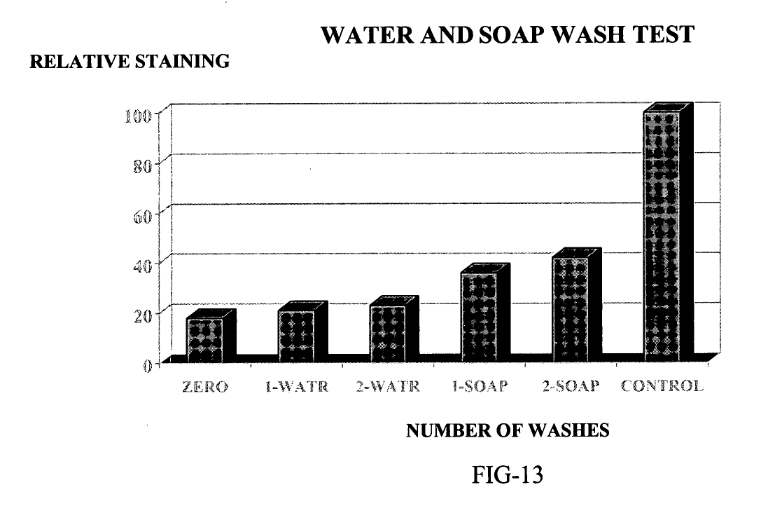

One formulation of the present invention is to a product that not only kills harmful microorganisms, but may also protect the skin from harmful and irritating substances. The antibacterial formulation contains ingredients which moisturize the skin while at the same time form an invisible and undetectable polymer film that cover the outer layer of the skin, sealing in many of the skin's natural moisturizers and sealing out undesirable contaminants and irritants. When applied as directed, the antibacterial formulation will last 4 to 6 hours without reapplication. As is shown in FIGS. 11-13 the product of the present invention confers protection even when washed with mild soaps and alcohol-based solvents. Once applied the dispersed droplets penetrate the outer skin layer (stratum corneum) to increase its lipid barrier against water borne irritants. The polysaccharide matrix forms a protective shield adherent to the oil-coated stratum corneum, thus, forming a gas impervious barrier against damaging environmental agents.

One antibacterial formulation of the present invention is a novel starch-oil suspension composite produced by a jet heating and pressure process. It is composed of a selected oils, starches, and polysaccharide polymers. Specific formulations have been developed to tailor lotions for maximal protection and increased skin support and moisturization. This was accomplished by blending process that disperses many different kinds of oil, including plant oils, mineral oil or petrolatum with high molecular weight starches. The particular blend of oils and starches used defines the resultant soft feel, and texture, and produce a highly skin-friendly support system unlike most detergent containing oil-in-water emulsions. Most importantly, these ingredients may be mixed without the use of any irritating surfactants or emulsifiers (surface-active agents) or chemical stabilizers present in most skin care products. The formulation produces a highly lubricious barrier, again, in contrast to most detergent containing oil-in-water emulsions. Equally important are the facts that the antibacterial of the present invention is formulation-insensitive to added active ingredients that require a stable pH for activity. This property makes it compatible with vitamins, anti-oxidants and skin rejuvenating ingredients.

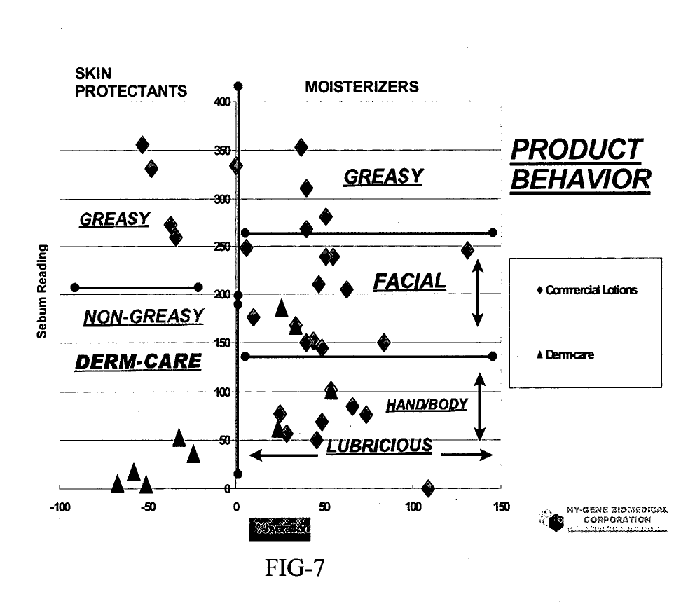

Extensive physical and human tests have been performed showing that the formulation of the present invention provides a protective physical polymer film on skin. The protective effect was compared with many leading marketed brands of skin protectants. The results of skin hydration measurements showing a protective effect (reduced loss of skin moisture) are summarized in FIG. 7. The formulation of the present invention outperformed all of the other tested products. In order to achieve a protective effect, most other skin protectants must coat the skin with a oil layer to be effective, thus, leaving a greasy oil residue on the skin. By contrast, the formulation of the present invention leaves no greasy oily residue on the skin. This was confirmed by laboratory tests using a Sebumeter device that measures residual oil on skin (see FIG. 7).

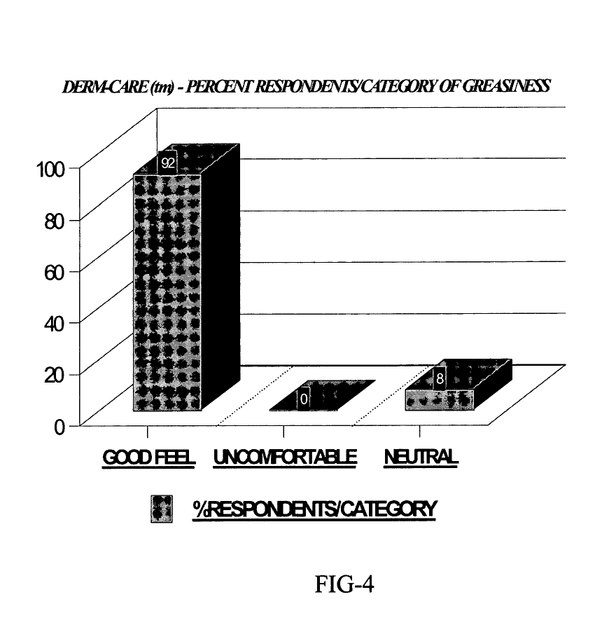

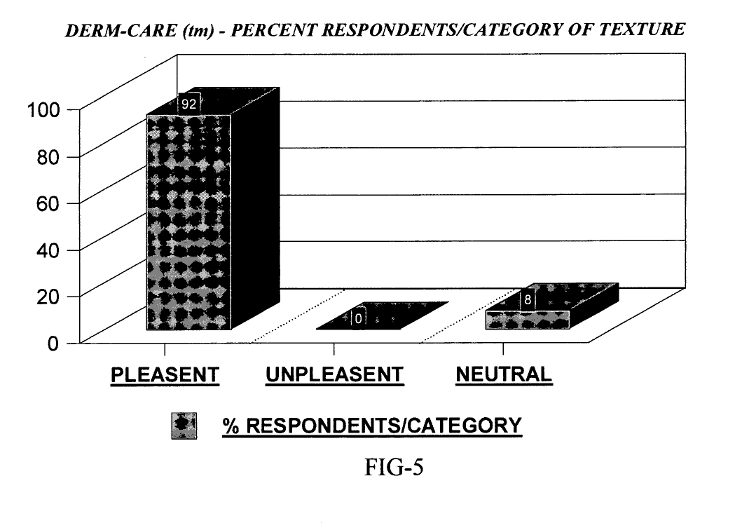

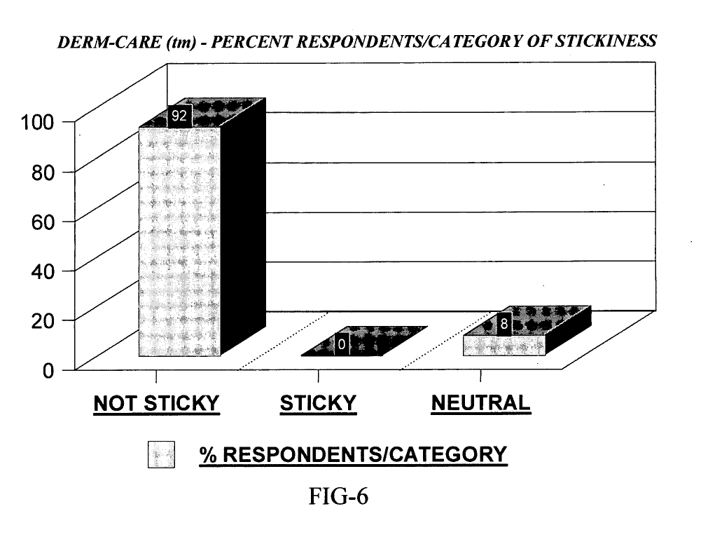

One formulation of the present invention is a skin moisturizing lotion that has been formulated with natural humectants to provide high performance moisturization and hydrating properties relative to other marketed skin moisturizer products. This product is also based on emulsion technology and utilizes a special polysaccharide-dimethicone stable oil-in-water lotion that is totally transparent and non-greasy, and is formulated to both moisturize and protect the skin with excellent feel to the user. FIGS. 4-6 show respondents grading of the formulation of the present invention in the categories of greasiness, texture and stickiness.

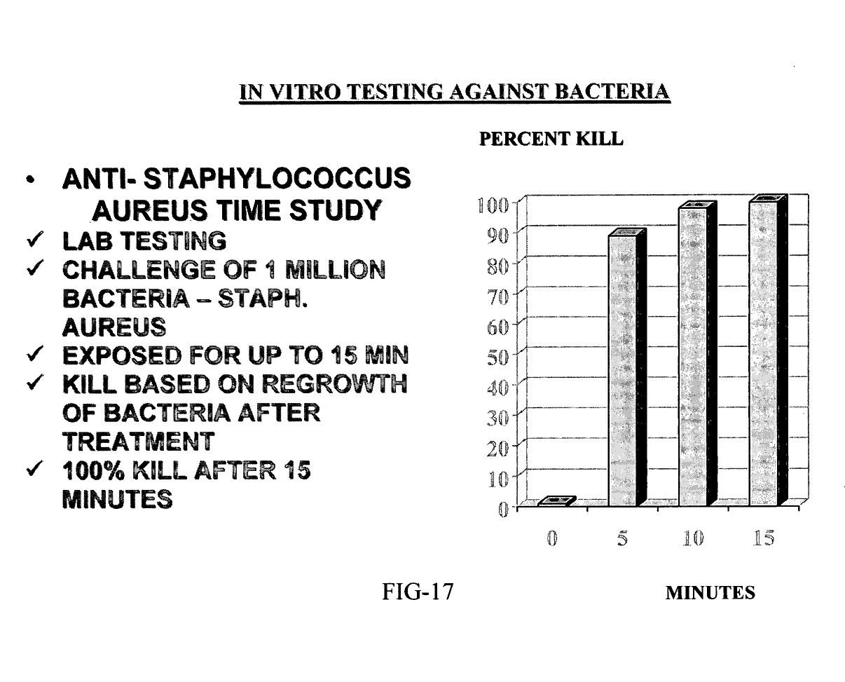

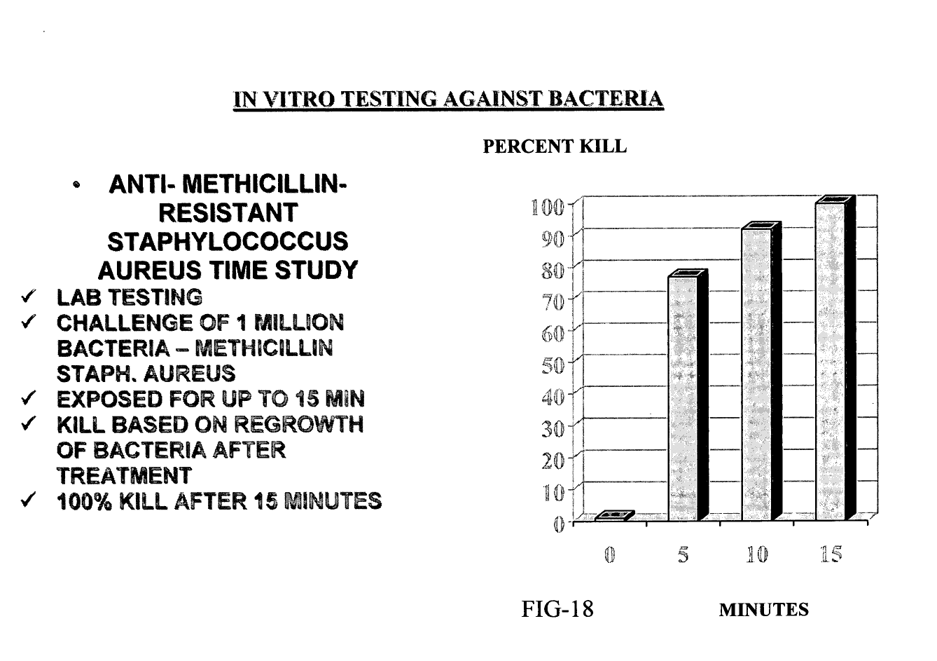

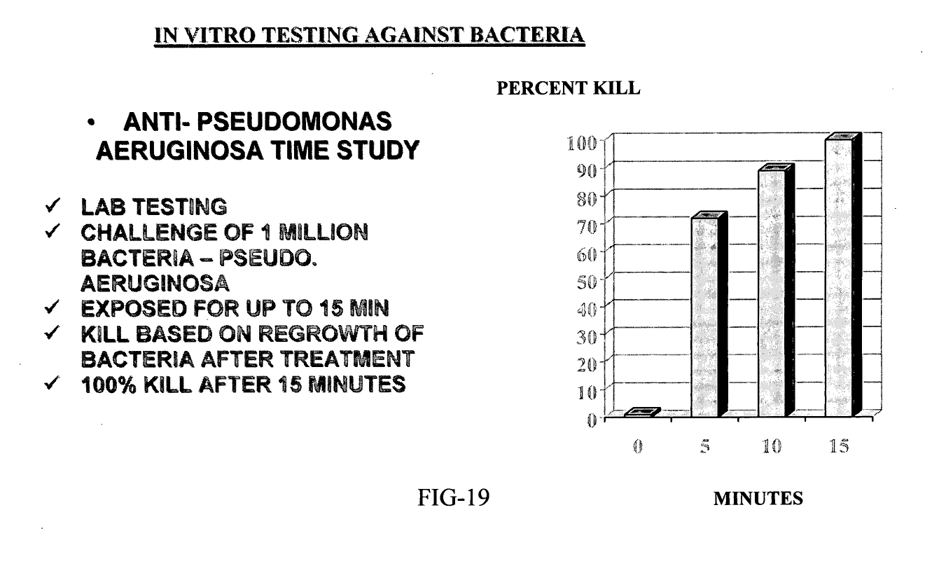

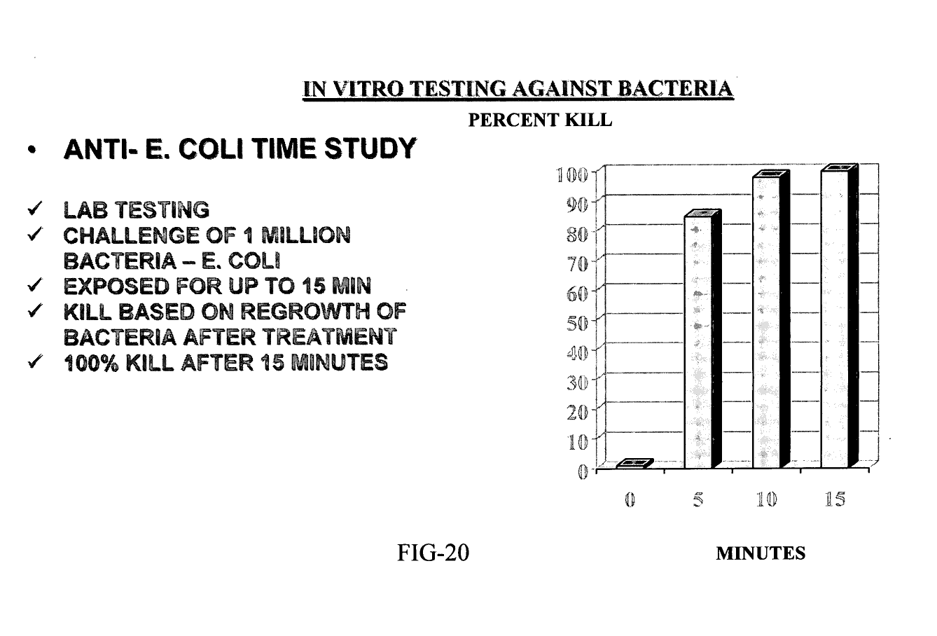

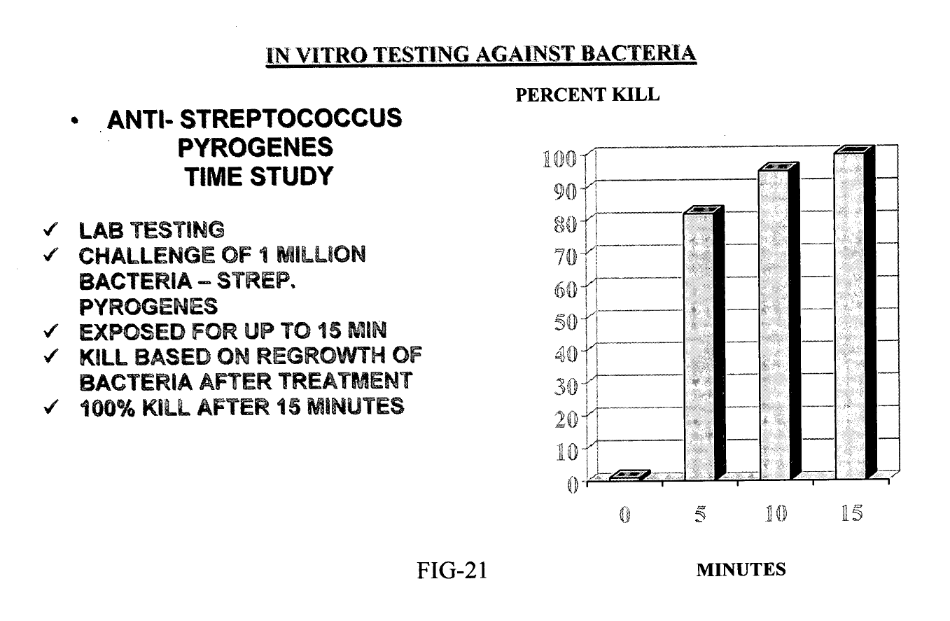

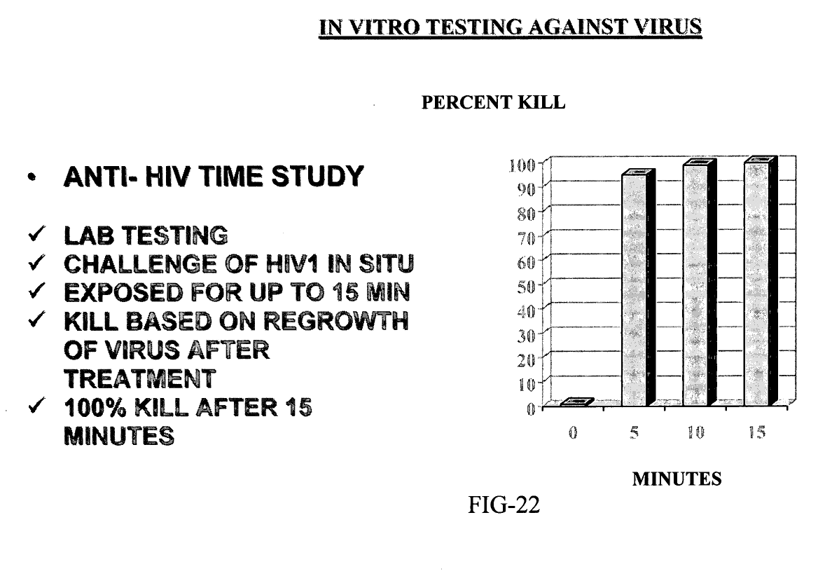

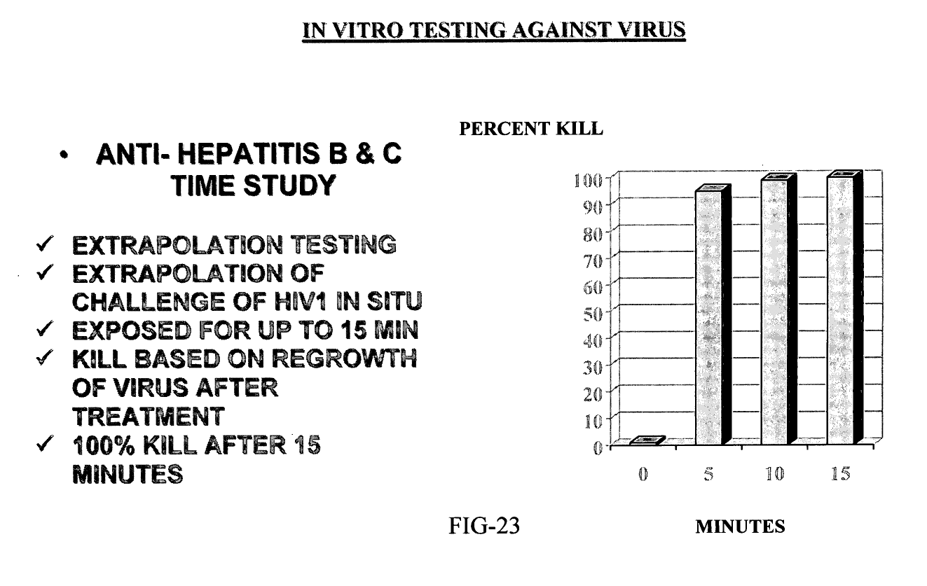

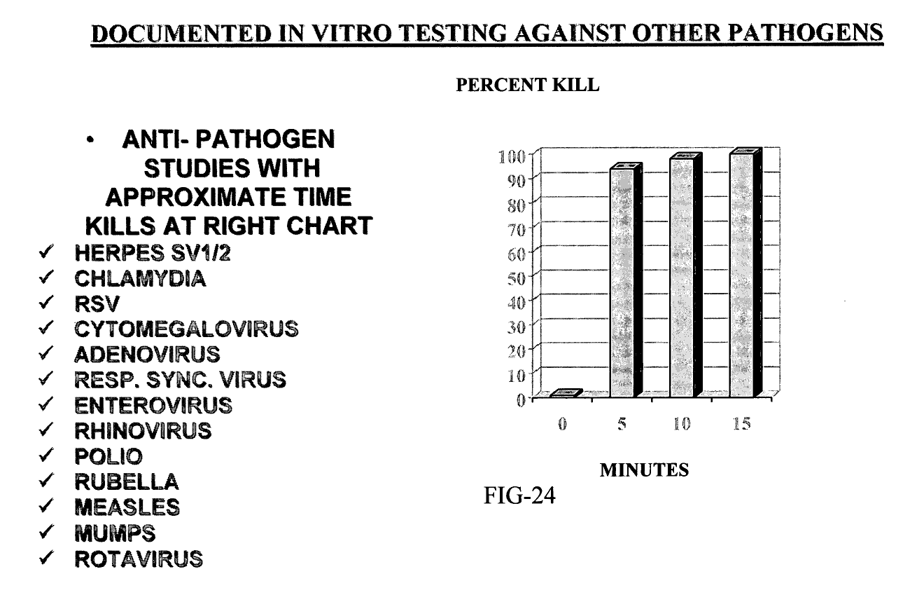

The antibacterial lotion of the present invention is especially designed to fight microbial pathogens. The active antimicrobials provide protection against a broad spectrum of pathogens such as infectious Staphylococcus aureus, Escherichia coli, Streptococci, HIV, E. coli, and Pseudomonas aeruginosa. See FIGS. 17-24, which show the pathogen percent kill rate with application of the formulation of the present invention.

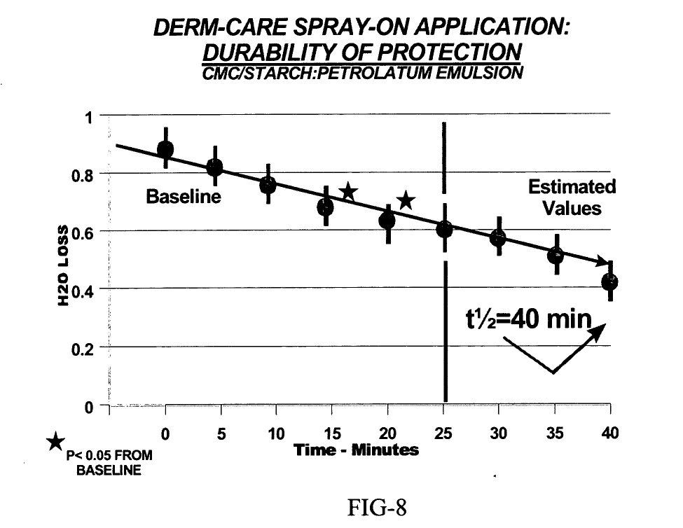

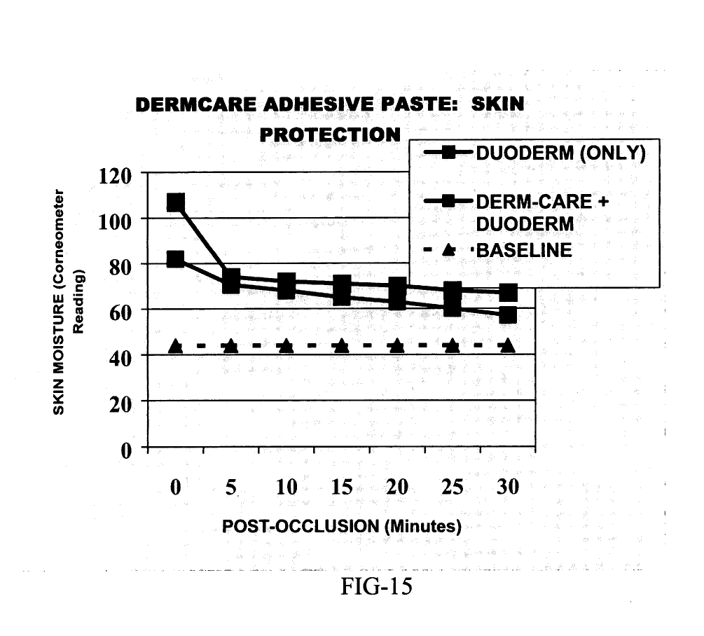

Products of the present invention may come in many different formulations. The durability of protection of a spray-on application is shown in FIG. 8. An adhesive paste formulation is evaluated for skin moisture in FIG. 15. Following is the ingredient list on the MSDS for the hand cleanser formulation of the present invention showing the ingredients with preferred weight percent and in parenthesis the range of weight percent that is possible to use:
Purified water—96.35% (85.0-99.5%)
N,N Bis (2-OH-ethyl) Lauramide—1.0% (0.25-5.0%)
Na C₁₄-C₁₆ Olefin Sulfonate—0.5% (0.05-3.0%)
Polyethylene Glycol (1-4) Lauryl Ether SO₄ (Na salt)—0.35% (0.03-3.5%)
Polyquaternium-7—0.30% (0.015-2.5%)
Chlorhexidine—1% (0.25-4.0%)
Plant fatty acids (optional)—0.25% (0.02-1.5%)
Antioxidant(s)—0.2% (0.05-1.0%)
Phenolic(s)—0.3% (0.015-1.75%)

The following is the ingredient list from the MSDS of the spray surface cleaner formulation of the present invention showing the ingredients with preferred weight percent and in parenthesis the range of weight percent that is possible to use:
Purified water—96.35% (85.0-99.5%)
Sodium metasilicate 0.5H₂O
Nonoxynol-9 or equal—0.25% (0.02-2.5%)
Butyl cellusolve—2.0% (0.05-5.5%)
Barquat 50 as 50% solution—0.60 (0.05-3.5%)
Barlox 12—0.25% (0.01-1.5%)
May contain—benzoyl peroxide—1.0% @q.s. (0.05-3.0%)
   The following may be added to the above formulation as a base for stabilization and for enhanced residual killing capacity of pathogens on surfaces showing the ingredients with preferred weight percent and in parenthesis the range of weight percent that is possible to use:
Waxy Starch OR other Polysaccharide (natural or synthetic)—1% (0.05-2.5%)
Petrolatum OR other mineral oil—0.5% (0.125-2.0%)
Other Plant/vegetable oils or extracts—0.15% (0.01-2.0%)
Pectin OR other viscosity stabilizer—0.05% (0.01-1.0%)
Xanthan OR other viscosity enhancer—0.05% (0.01-1.0%)
d-α-tocopherol or other antioxidant or stabilizer—0.05% (0.01-1.0%)
Benzoic Acid—0.1%—OPTIONAL (0.01-1.0%)
Boric Acid—0.1%—OPTIONAL (0.01-1.0%)

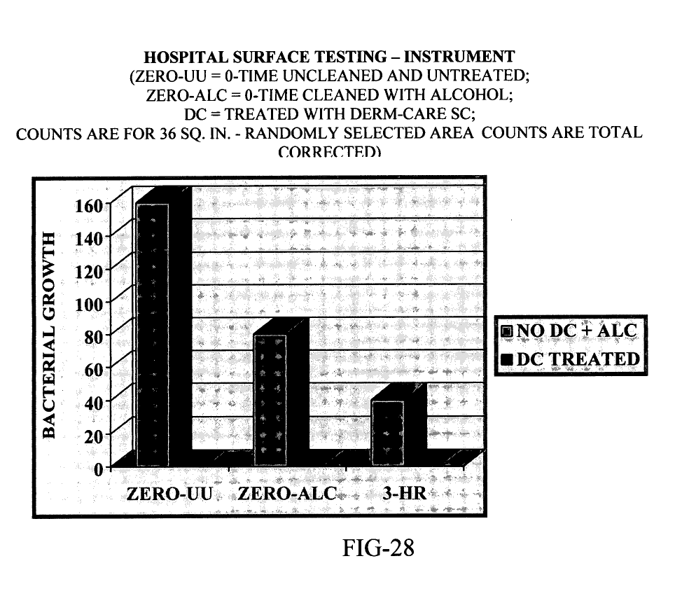

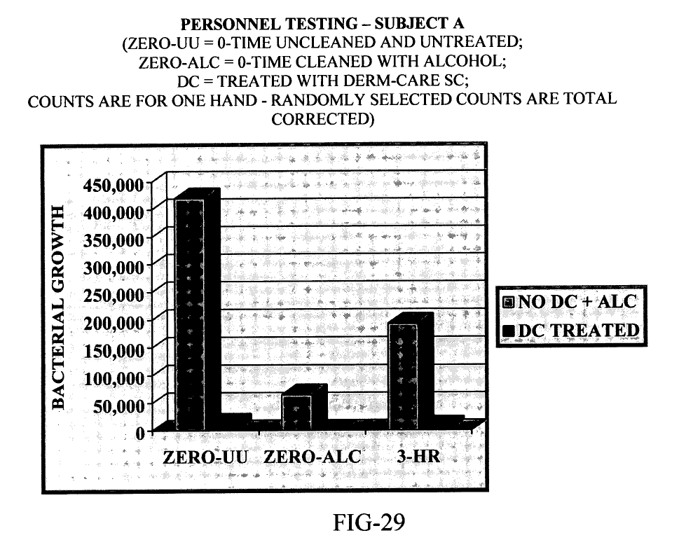

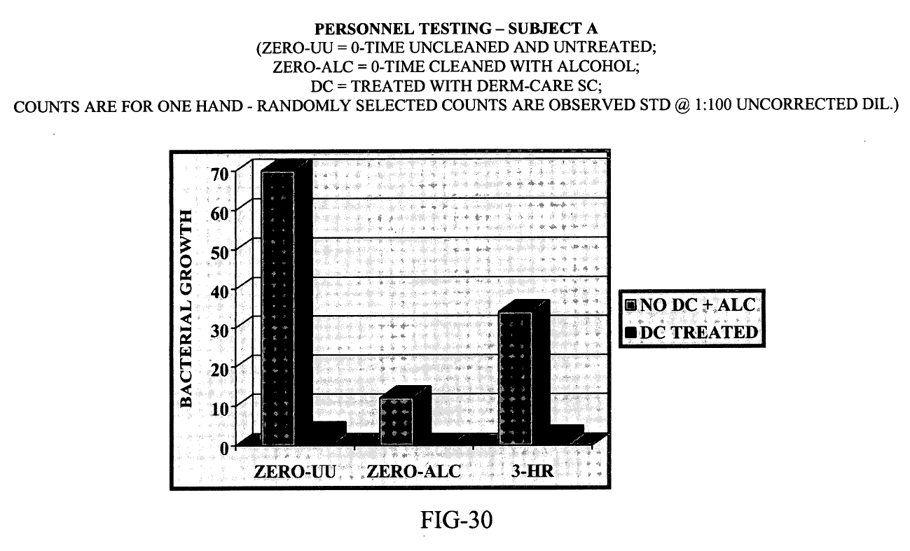

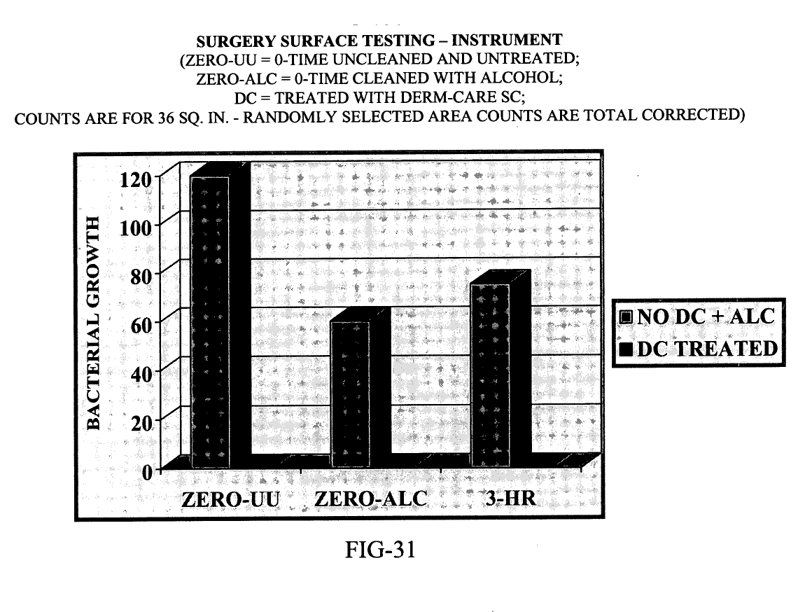

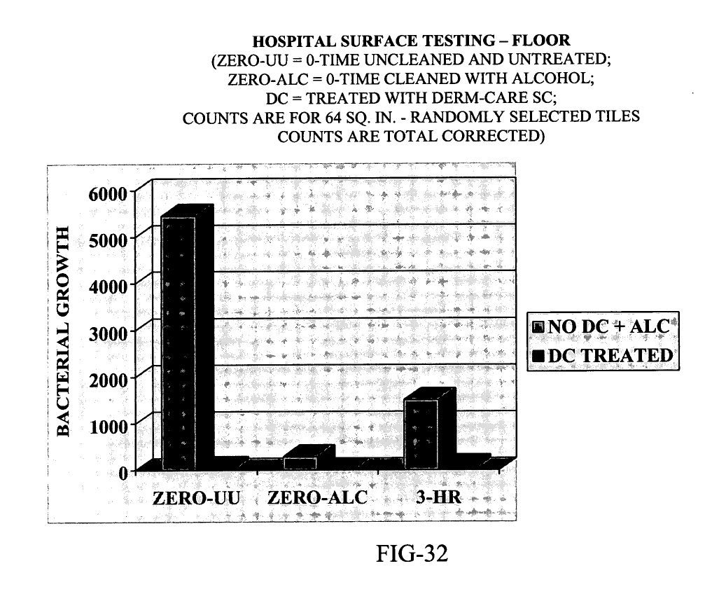

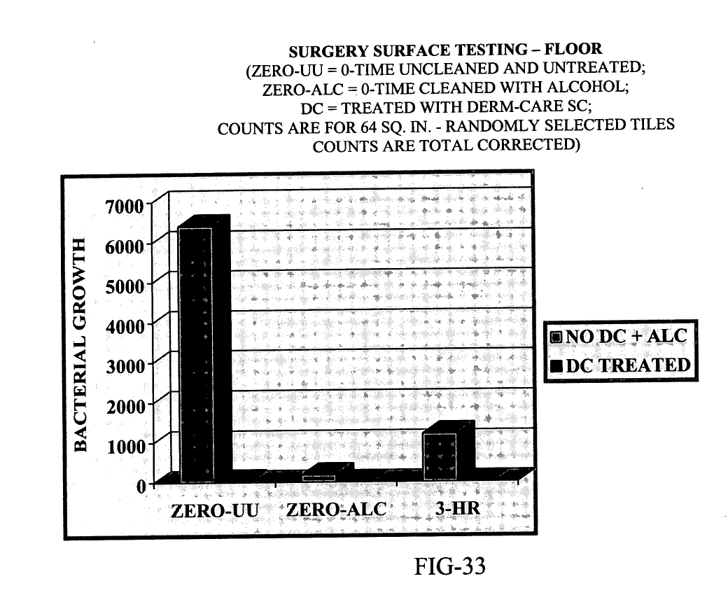

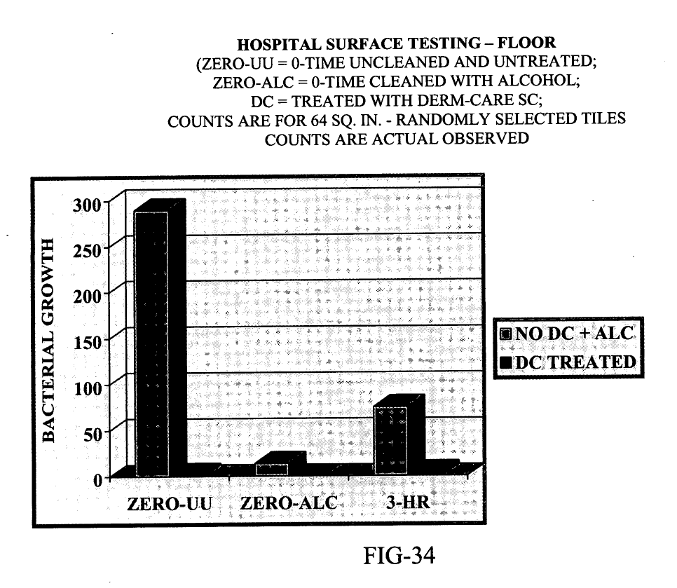

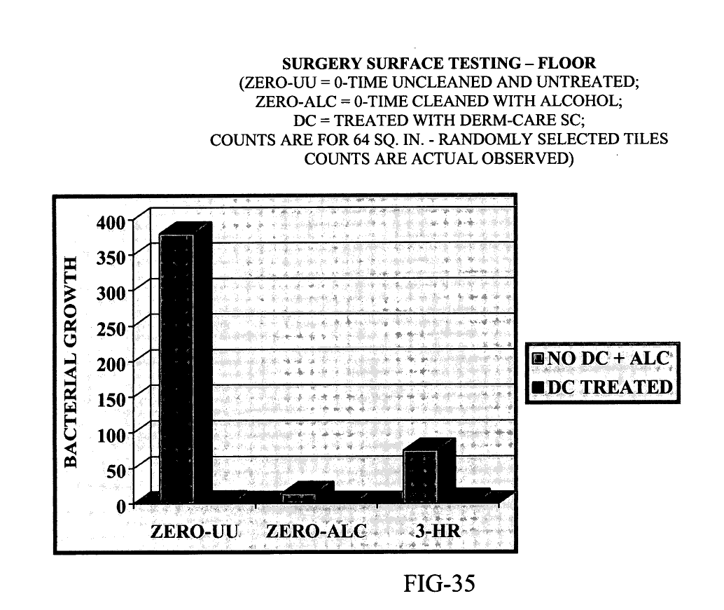

FIGS. 28-35 show, via bar graphs, bacterial growth counts on instruments in a general hospital setting, corrected bacterial growth counts on personnel hands in a hospital setting, uncorrected bacterial growth counts on personnel hands in a hospital setting, bacterial growth counts on surfaces in a surgical setting, corrected bacterial growth counts on floor surfaces in a general hospital setting, corrected bacterial growth counts on floor surfaces in a surgical setting, uncorrected bacterial growth counts on floor surfaces in a general hospital setting and uncorrected bacterial growth counts on floor surfaces in a surgical setting (respectively). The bacterial counts were taken of non-cleaned and untreated instruments, hands, surfaces and floors, taken after being cleaned with alcohol and taken after treatment with the spray formulation of the present invention.

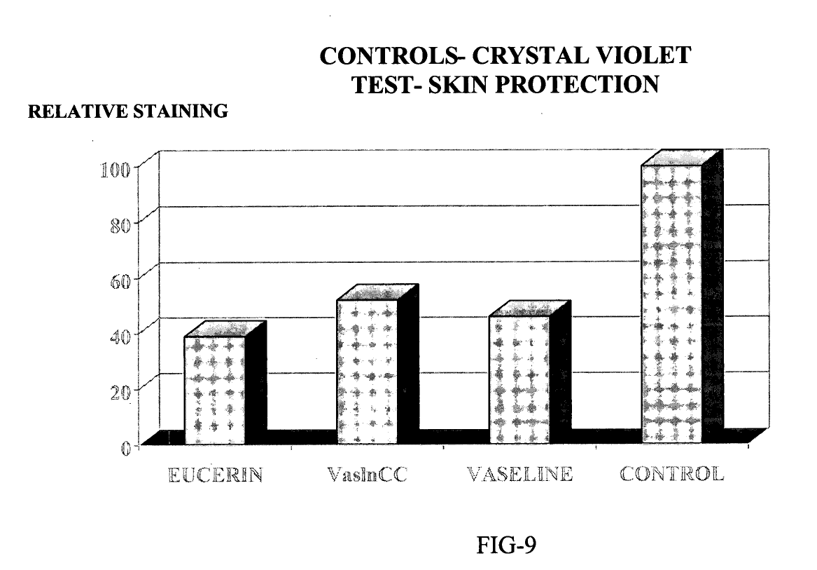

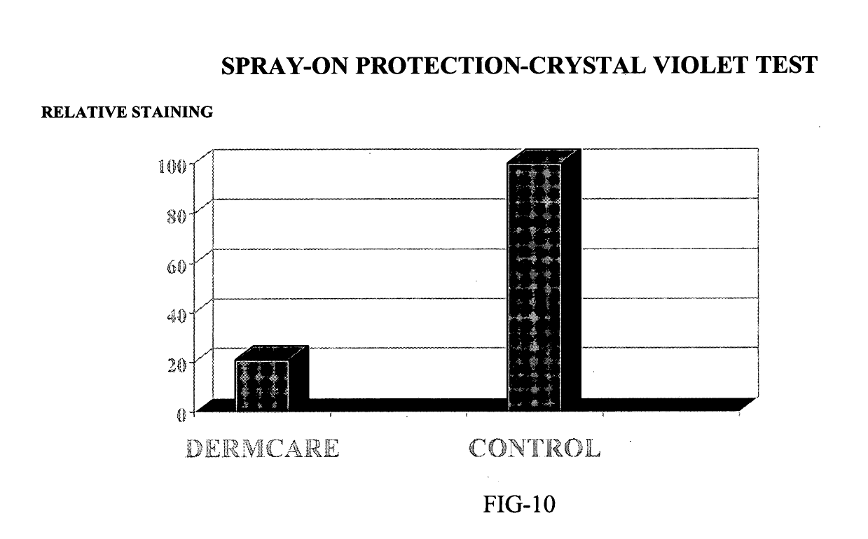

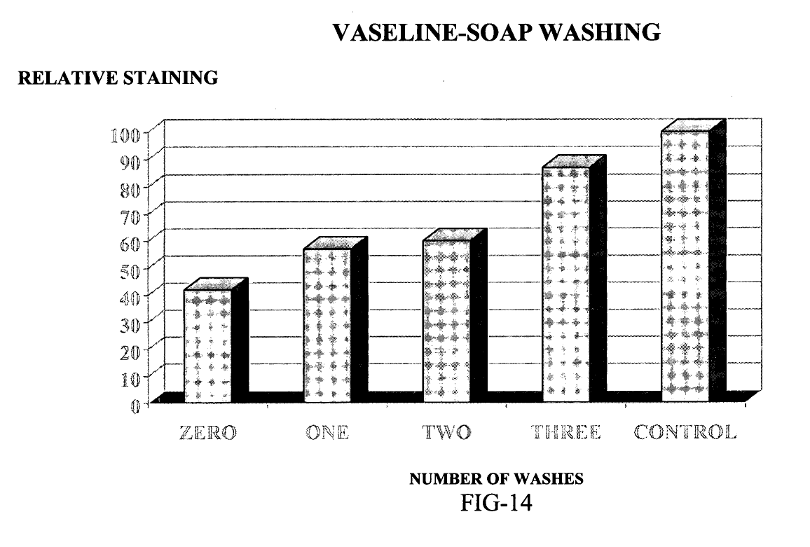

The present invention is also to a validation and monitoring kit and method for determining if employees are protected and following the proper protocol of application of products of the present invention. The kit is contains a small bottle with a removable cap. Under the cap is a wick, which resembles a large pen marker. The bottle contains a blue dye (crystal violet) material in water. Once the product of the present invention has been applied as directed and dried, the applicator tip may be touched to any topical surface and pulled along the surface for about 1 to 1½ inches. The blue dye is left in place for about 5-8 seconds and then wiped with a tissue. If the product of the present invention has covered the area properly, no stain color should be present. If the area is not covered a streak of blue stain will remain indicating the barrier has not been applied. This test may be randomly applied to any part of the hands after repeated applications so that compliance testing may be accomplished without consideration for predictable routine application to the same area, e.g. the back of the hand versus random testing to the back of the hand, side of the finger, base of the thumb, etc. FIGS. 9-14 exemplify the crystal violet staining with the product of the present invention.

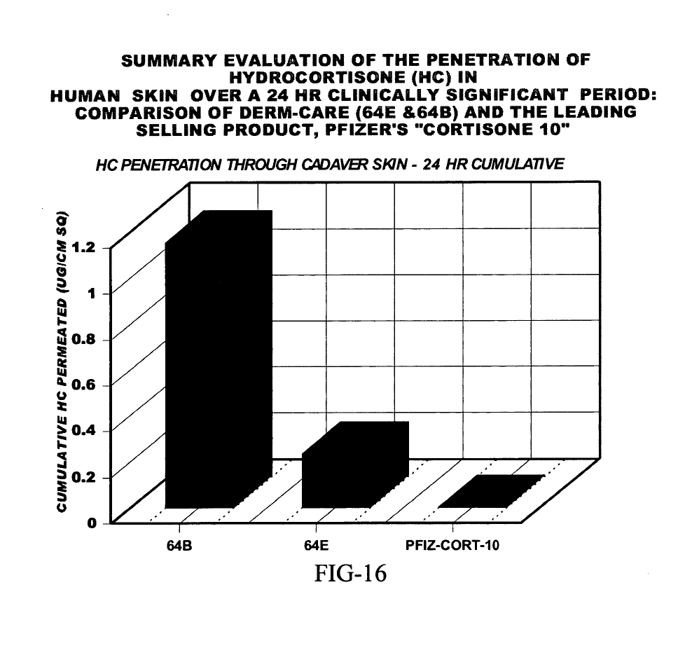

FIG. 16 exemplifies the ability of formulations of the present invention, after forming a barrier on the skin, to deliver significantly more drug (in this case hydrocortisone), locally across the skin, than a product in the prior art.

While example embodiments of the invention have been illustrated and described, various modifications and combinations can be made without departing from the spirit and scope of the invention. Although the present invention has been described in relation to health care (hospital) and food preparation environments, the disclosed formulations may be used in any environment. Modifications, combinations, and equivalents to the formulations and system of the present invention are intended to be covered and claimed.

1. A system for managing pathogens and irritants comprising the steps of:
a. washing skin with an antibacterial hand soap;
b. applying an antibacterial skin barrier lotion; and
c. spraying an antibacterial surface cleaner on hard surfaces in an area.

2. A validation and monitoring kit used to determine if one has complied with the application of an antibacterial hand soap or a skin barrier lotion, said validation and monitoring kit comprising: a bottle with a removable cap, a wick, and a dye diluted in water.

3. An emulsion formulation for cleansing the skin comprising: purified water in an amount of about 85 to about 99.5 percent by weight, N,N-bis-(2-OH-ethyl) lauramide in an amount of about 0.25 to about 5 percent by weight, a sodium sulfonate of C₁₄₋₁₆ olefin in an amount of about 0.05 to about 3 percent by weight, polyethylene glycol lauryl ether sodium sulfuric acid in an amount of about 0.03 to about 3.5 percent by weight, polyquaternium-7 in an amount of about 0.015 to about 2.5 percent by weight, cholorhexidine in an amount of about 0.25 to about 4 percent by weight, an antioxidant in an amount of about 0.05 to about 1 percent by weight and phenolic in an amount of about 0.015 to about 1.75 percent by weight.

4. The emulsion formulation of claim 3 additionally comprising a plant fatty acid in an amount of about 0.02 to about 1.5 percent by weight.

5. An emulsion formulation for topical application to the skin comprising: purified water in an amount of about 80 to 99.5 percent by weight, a group selected from the group consisting of waxy starch and a polysaccharide in an amount of about 0.5 to about 15 percent by weight, a group selected from the group consisting of petrolatum and any other mineral oil in an amount of about 0.125 to about 12 percent by weight, other plant or vegetable oils or extracts in an amount of about 0.01 to about 8 percent by weight, a viscosity stabilizer in an amount of about 0.01 to about 3 percent by weight, a viscosity enhancer in an amount of about 0.01 to about 3 percent by weight, an antioxidant or stabilizer in an amount of about 0.01 to about 5 percent by weight, MgCl₂ in an amount of about 0.01 to about 1 percent by weight, methyl paraben in an amount of about 0.01 to about 1 percent by weight, propyl paraben in an amount of about 0.01 to about 1 percent by weight, citricidal in an amount of about 0.01 to about 3 percent by weight, benzalkonium chloride in an amount of about 0.01 to about 2 percent by weight, and tri-N-butyl PO₄ in an amount of about 0.01 to about 1 percent by weight.

6. An emulsion formulation of claim 5 additionally comprising benzoic acid in an amount of about 0.01 to about 1 percent by weight.

7. An emulsion formulation of claim 5 additionally comprising boric acid in an amount of about 0.01 to about 1 percent by weight.

8. An emulsion formulation of claim 5 additionally comprising benzoyl peroxide in an amount of about 0.05 to about 3 percent by weight.

9. A spray formulation for application to surfaces comprising: purified water in an amount of about 85 to about 99.5 percent by weight, sodium metasilicate, nonoxynol-9 in an amount of about 0.02 to about 2.5 percent by weight, butyl cellusolve in an amount of about 0.05 to about 5.5 percent by weight, barquat 50 in an amount of about 0.05 to about 3.5 percent by weight, and barlox 12 in an amount of about 0.05 to about 3 percent by weight.

10. An emulsion formulation of claim 9 additionally comprising benzoyl peroxide in an amount of about 0.05 to about 3 percent by weight.

11. A method of validating and monitoring to see if one has complied with the application of an antibacterial hand soap or a skin barrier lotion comprising the steps of: touching an applicator tip containing a dye to any topical surface, pulling the applicator tip along said surface for about 1 to 1½ inches, drying said dye for about 5-8 seconds, wiping said surface with a tissue and observing whether there is a colored stain on said surface.

12. The method of claim 11, wherein said topical surface is human hands.

13. The method of claim 11, wherein said dye is a blue-colored dye.

14. The method of claim 13, wherein said blue dye is crystal violet.
